(19) United States
(12) Patent Application Publication
(10) Pub. No.: US 2009/0318552 A1
(43) Pub. Date: Dec. 24, 2009

(54) Pharmaceutical composition comprising shikonin derivatives from lithospermum erythrorhizo dor treating or preventing diabetes mellitus and the use thereof

(76) Inventors: Ji Ho Park, Seoul (KR); Sun Yeou Kim, Yongon-si (KR); Tong Ho Kang, Seoul (KR); Eun Ju Hwang, Yongon-si (KR); Chul Hoon Kang, Suwon-si (KR)

Correspondence Address: Olson & Cepuritis, LTD., 20 NORTH WACKER DRIVE, 36TH FLOOR, CHICAGO, IL, 60606, US

(21) Appl. No.: 12/456,171

(22) Filed: Jun. 12, 2009

Related U.S. Application Data
(63) Continuation-in-part of application No. 12/312,943.

(51) Int. Cl. A61K 31/216; C07C 69/007

(52) U.S. Cl. 514/546; 560/129

(57) ABSTRACT

The present invention relates to a composition for preventing and treating diabetes mellitus that includes a shikonin compound as an active ingredient and to a use thereof. More particularly, the present invention relates to a composition for preventing and treating diabetes mellitus that includes isobutyryl shikonin, β,β-dimethylacryl shikonin, isovaleryl shikonin, or α-methyl-n-butyryl shikonin as an active ingredient and to a use thereof. The composition of the present invention exhibits antidiabetic effects by blocking the K_{ATP} ion channels (ATP-sensitive potassium ion channels) of beta (β) cells in the pancreas and increasing the concentration of calcium ions to promote the secretion of insulin, and thus can be used as a medicinal pharmaceutical, food additive, or health functional food for preventing and treating diabetes mellitus.

### CROSS-REFERENCE TO RELATED APPLICATION

This application is a continuation-in-part of U.S. Ser. No. 12/312,943 filed on Jun. 1, 2009, which is the U.S. National Phase of PCT/KR2006/005414.

### TECHNICAL FIELD

The present invention relates to a pharmaceutical composition comprising a shikonin compound, and the use thereof, for the prevention and treatment of diabetes mellitus.

### BACKGROUND OF THE INVENTION

Diabetes can be classified into two major types. One is insulin-dependent diabetes mellitus (type I), which corresponds to about 10% of diabetes patients. Since it occurs in a younger age group of 20 years or younger, it is thought to be genetic, and it is also referred to as juvenile diabetes. This type generally causes weight loss and is likely to cause ketonacidosis. It is possible to remedy this type of administering insulin. This type is often found in an acute form and incurs more severe symptoms in children than in adults.

The other type is insulin-independent diabetes mellitus (type II), which mainly occurs in people aged 40 years or older. Caused by lack of exercise, obesity, overeating, stress, etc., the receptivity to insulin is lowered in peripheral tissues, such as the muscle or fat tissues, causing disturbances in sugar metabolism over a long period of 4 to 5 years. Type II diabetes can be cured in about 50 to 80% of all cases, if the patient achieves weight loss by means of diet therapy and exercise therapy, and does not pose a risk of death even without insulin shots, and thus type II diabetes is referred to as insulin-independent diabetes.

With the drastic increase in the obese population in recent times, the prevalence of type II diabetes has greatly increased. According to recent reports, a decrease in insulin action in a normal person causes the beta (β) cells of the pancreas to release more insulin correspondingly and supplement the decrease, but in the case of type II diabetes, a decrease in insulin action also causes a decrease in insulin release, to result in the disease (Kahn, B. B., Cell 92, pp. 593-596, 1998; Kahn, B. B., Nature Genet. 20, pp. 223-225, 1998).

It is also known that type II diabetes is in direct correlation with the K_{ATP} ion channels (ATP-sensitive potassium ion channels) of the beta cells of the pancreas (Tarasov, A. et al., Diabetes., 53(3), pp. S113-S122, 2004). When the blood glucose level is low, the K_{ATP} ion channels of the beta cell in the pancreas are opened, and the K⁺ ions move out of the cells, resulting in membrane hyperpolarization. Thus, the Ca²⁺ ion channels are closed, and insulin is not released. However, when the blood glucose level is high, glucose is moved into the beta cells, and as the levels of ATP in the cells are increased, the K_{ATP} ion channels are closed. This results in depolarization, and as Ca²⁺ ions move into the cells through the Ca²⁺ ion channels, there is a corresponding release of insulin. In the case of a diabetic, however, the above activities are not properly implemented, resulting in diabetes. Glibenclamide, which is a type of a sulfonylurea, is a representative means for forcibly closing the K_{ATP} ion channels and is currently used as a diabetes remedy.

While various oral hypoglycemic agents are currently used in clinics to treat diabetes, these cause various side effects, such as hypoglycemia, hepatotoxicity, weight gain, lactic acidosis, etc. Thus, for the treatment of diabetes, there is a need to develop a preparation or a functional ingredient that does not incur side effects and offers various mechanisms, and there is a demand for developing a diabetes remedy by analyzing natural materials that contain various constituents.

Lithospermum erythrorhizon (Siebold & Zuccarini), which belongs to the family Boraginaceae, is distributed over Japan, Manchuria, and China. While it was once found in mountains and fields all across Korea, it is now seldom found, although it is often grown in Jindo of the Jeollanam-do province.

The root of the Lithospermum erythrorhizon contains shikonin, which is a derivative of naphthoquinone having a reddish color, acetyl shikonin, isobutyryl shikonin, β,β-dimethylacryl shikonin, isovaleryl shikonin, α-methyl-n-butyryl shikonin, and β-hydroxyisovaleryl shikonin, etc. In oriental medicine, the root is used as a medicine for strengthening the stomach, increasing energy, treating jaundice, treating gonorrhea, treating scabies, increasing vigor, neutralizing toxins, alleviating fever, facilitating urination and defecation, lowering body temperature, treating tumefaction, treating burns, treating frost bite, treating eczema, treating blisters, and preventing conception, etc., while local remedies have used the root as an anti-aging drug. It is also known to have anti-inflammatory, antimicrobial, and antiviral effects.

Shikonin compounds are also used as ointments for treating bruises and burns, cosmetic ingredients, and antibiotics, while recent studies on shikonin compounds, which are contained in the root of the Lithospermum erythrorhizon, have reported on their anticancer (Xin, C. et al., Phytotherapy Research 16(3), pp. 199-209, 2002) and antioxidant (Weng, X. C. et al., Food Chemistry 69(2, pp. 143-146, 2000) effects. Also, while the intracellular signal transfer of shikonin in 3T3-L1 fat cells is different from that of insulin, it has been shown in a recent study that similar actions to insulin are exhibited in terms of absorbing glucose (Kamei, R. et al, Biochem. Biophys. Res. Commun., 292, pp. 642-651, 2002).

Isobutyryl shikonin, β,β-dimethylacryl shikonin, isovaleryl shikonin, α-methyl-n-butyryl shikonin and β-hydroxyisovaleryl shikonin are compounds included in the root of the Lithospermum erythrorhizon, but there is as yet no research on these compounds published either at home or abroad, and in particular, there is as yet no disclosure or teaching regarding their antidiabetic effects.

Therefore, the present inventors have completed the present invention by confirming that isobutyryl shikonin, β,β-dimethylacryl shikonin, isovaleryl shikonin and α-methyl-n-butyryl shikonin inhibit the K_{ATP} ion channels of beta cells in the pancreas.

### SUMMARY OF THE INVENTION

An objective of the present invention is to provide a pharmaceutical composition for preventing and treating diabetes mellitus and usage thereof that contains a shikonin derivative compound as an active ingredient.

To achieve the above objective, the present invention provides a pharmaceutical composition for preventing and treating diabetes mellitus that contains a shikonin compound represented by the following general formula (1) as an active ingredient and includes a pharmaceutically acceptable carrier, excipient, or diluent:

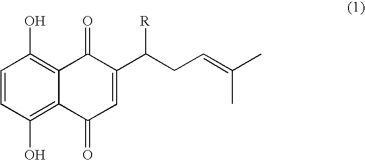

wherein,

R is a member of the group consisting of OCOCH(CH₃)₂, OCOCH═C(CH₃)₂, OCOCH₂CH(CH₃)₂, and OCOCH(CH₃)CH₂CH₃.

The shikonin compound represented by the general chemical formula (1) includes isobutyryl shikonin, β,β-dimethylacryl shikonin, isovaleryl shikonin, or α-methyl-n-butyryl shikonin.

The present invention provides a use of a composition that contains a shikonin compound represented by general chemical formula (1) as an active ingredient and includes a pharmaceutically acceptable carrier, excipient, or diluent for preparing a pharmaceutical intended for treating and preventing diabetes mellitus.

Also, the present invention provides a method of treating or preventing diabetes mellitus by administering to a person or a mammal a suitable amount of a shikonin compound represented by general chemical formula (1) and a pharmaceutically acceptable carrier or excipient.

A method of obtaining a Lithospermum erythrorhizon extract and methods of isolating and refining shikonin compounds will be described below in more detail.

The Lithospermum erythrorhizon extract of the present invention can be obtained by grinding a Korean-grown Lithospermum erythrorhizon, drying it in a freeze-dryer, storing in a refrigerator at a temperature of 0° C. or lower, preferably at −20° C., and then performing ultrasonic extraction using water, a lower alcohol of C₁ to C₄, or a mixed solvent thereof, preferably ethanol, in a quantity of 1 to 20 times, preferably 3 to 10 times the weight (kg) of the Lithospermum erythrorhizon, for a period of 5 minutes to 5 hours, preferably 1 hour, to obtain the Lithospermum erythrorhizon extract.

For the Lithospermum erythrorhizon extract thus obtained, the organic solvent was dried under a reduced pressure, and the residue was suspended in water, to be divided, using a chloroform-ethanol (2:1) solution and purified water, into a water layer and a chloroform-ethanol fraction. By performing HPLC for the chloroform-ethanol (2:1) fraction, isobutyryl shikonin, β,β-dimethylacryl shikonin, isovaleryl shikonin, or α-methyl-n-butyryl shikonin, of Chemical Formulas 1 to 4 can be isolated.

The compounds of the present invention represented by the general formula (1) above can be prepared as pharmaceutically acceptable salts and solvates using typical methods in the related art.

Acid addition salts formed by free acids can be utilized as pharmaceutically acceptable salts. An acid addition salt is prepared by a typical method, for example by dissolving the compound in an excess amount of an aqueous acid solution, and then precipitating the salt using a water-soluble organic solvent such as methanol, ethanol, acetone or acetonitrile. Equivalent moles of the compound and the acid or alcohol (e.g. glycol monomethyl ether) in the water can be heated, and the mixture can be subsequently evaporated and dried, or the sedimented salts can be suction-filtered.

Here, the free acid can be an organic acid and an inorganic acid, where the inorganic acid can be hydrochloric acid, phosphoric acid, sulfuric acid, nitric acid, threaric acid, etc., and the organic acid can be methanesulfonic acid, p-toluenesulfonic acid, acetic acid, trifluoroacetic acid, citric acid, maleic acid, succinic acid, oxalic acid, benzoic acid, tartaric acid, fumaric acid, manderic acid, propionic acid, citric acid, lactic acid, glycolic acid, gluconic acid, galacturonic acid, glutamic acid, glutaric acid, glucuronic acid, aspartic acid, ascorbic acid, carbonylic acid, vanillic acid, hydroiodic acid, etc.

Furthermore, pharmaceutically acceptable metal salts can be made using bases. Alkali metals or alkaline earth metal salts can be obtained, for example, by dissolving the compound in an excess amount of an alkali metal hydroxide or alkaline earth metal hydroxide solution, filtering the undissolved compound salt, and then evaporating and drying the remainder. Here, it is especially desirable, in terms of preparing a pharmaceutical, to use sodium, potassium, or calcium salts for the metal salts, and corresponding silver salts can be obtained by reacting the alkali metal or alkaline earth metal salt with a suitable silver (e.g. silver nitrate).

The pharmaceutically acceptable salts for the general formula (1) above include, unless indicated otherwise, salts of acidic or basic groups that can exist within the compound of the general formula (1). For example, the pharmaceutically acceptable salts include sodium, calcium, and potassium salts of the hydroxyl group, while other pharmaceutically acceptable salts of the amino group include hydrogen bromide, sulfuric acid salts, hydrogen sulfuric acid salts, phosphate salts, hydrogen phosphate salts, dihydrophosphate salts, acetates, succinates, citrates, tartarates, lactates, mandelates, methane sulfonates (mesylates), p-toluenesulfonates (tosylates), etc, which can be prepared by methods and procedures well known in the art.

The present invention also provides a pharmaceutical composition containing a shikonin compound for preventing and treating diabetes mellitus prepared using the method described above.

In the composition of the present invention, a preferable content of the shikonin compound is 0.01˜99.9%, more preferably 0.1˜90%. However, the composition above is not thus limited, and the content may vary according to the condition of patient, the type of disease, and the progress of disease.

The composition containing a shikonin compound can further include a suitable carrier, excipient, and diluent commonly used in preparing pharmaceutical compositions.

The composition containing a compound according to the present invention can be prepared by a typical method in the form of an orally administered pharmaceutical, such as a powder, a grain, a tablet, a capsule, a suspension, an emulsion, a syrup, an aerosol, etc., or as an externally administered pharmaceutical, a suppository, or a sterile injectable solution, while the carriers, excipients, and diluents that can be included in the composition containing the extract can include, for example, lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. When formed as a pharmaceutical, a commonly used diluent or excipient can be used, such as a filler, an expanding agent, a binder, a wetting agent, a disintegrating agent, a surfactant, etc. A solid preparation for oral administration includes a tablet, a pill, a powder, a grain, a capsule, etc., where the solid preparation is prepared by mixing at least one or more excipients, such as starch, calcium carbonate, sucrose or lactose, gelatin, etc., to the extract. In addition to simple excipients, lubricants such as magnesium stearate talc are also used. A liquid preparation for oral administration includes a suspension, a solution, an emulsion, a syrup, etc., where various excipients can be included, other than the commonly used simple diluents of water and liquid paraffin, such as a wetting agent, a sweetener, an aromatic, a preservative, etc., for example. A preparation for non-oral administration includes a sterilized aqueous solution, a non-aqueous solution, a suspension, an emulsion, a freeze-dried pharmaceutical, and a suppository. The non-aqueous solution and the suspension can be propylene glycol, polyethylene glycol, a vegetable oil such as olive oil, an injectable ester such as ethyl oleate, etc. The base for a suppository can be witepsol, macrogol, tween-61, cocoa butter, laurin butter, glycergelatin, etc.

The preferred amount of the compound of the present invention actually administered may vary according to the condition and body weight of the patient, the severity of the disease, the form of the pharmaceutical, and the route and duration of administration, but may be selected appropriately by the skilled person. However, to obtain the preferred effects, it is desirable to administer the extract of the present invention in a daily dose of 0.01 mg/kg to 10 g/kg, preferably 1 mg/kg to 1 g/kg. The dose can be administered once a day or can be divided and administered several times a day. Thus, the dosage described above is not intended to limit the present invention in any way.

The pharmaceutical composition of the present invention can be administered to a mammal, such as a rat, mouse, domestic animal, human being, etc., via various routes. All modes of administration are contemplated, for example, administration can be made orally, rectally or by intravenous, intramuscular, vaginal epidural or intracerebroventricular injection.

The compound of the present invention is provided as a food product that contains other food ingredients in addition to the above composition effective for preventing and treating diabetes mellitus.

The food product includes confectionery, sugars, ice cream products, dairy products, meat products, fish products, tofu products or jelly products, edible oils and fats, noodles, tea products, beverages, special nutrient foods, health care foods, seasoning foods, ice, ginseng products, gimchi products, jerkys, and other food products.

The form of the food product includes powder, grain, tablet, capsule, liquid, or beverage forms.

Also, the present invention provides a food additive that contains the above compound effective for preventing and improving diabetes mellitus.

The composition of the present invention can additionally include any one or more of an organic acid, such as citric acid, fumaric acid, adipic acid, lactic acid, malic acid, etc.; a phosphate, such as sodium phosphate, potassium phosphate, acid pyrophosphate, polyphosphate (polymerized phosphate); and a natural antioxidant, such as polyphenol, catechin, α-tocopherol, rosemary extract, licorice root extract, chitosan, tannic acid, phytic acid, etc.

The above composition can be in the form of a highly-concentrated solution of 20 to 90%, or a powder, or a grain.

Similarly, the composition of the present invention can additionally include any one or more of lactose, casein, dextrose, glucose, sucrose and sorbitol.

The form of the food additive includes a powder, a grain, a tablet, a capsule, or a liquid.

Also, the present invention provides a method of using the above food additive in which the food additive is added to a food product as a disinfectant, a spice, a seasoning, various nutrients, a vitamin, a mineral (an electrolyte), a flavoring agent such as a synthetic flavoring agent and a natural flavoring agent, a coloring agent and an improving agent (cheese, chocolate, etc.), pectic acid and a salt thereof, alginic acid and a salt thereof, an organic acid, a protective colloidal thickening agent, a pH controlling agent, a stabilizing agent, a preservative, glycerin, alcohol, a carbonizing agent as used in carbonated beverages, etc., or as a critical ingredient of the food product. Here, the food additive can be added to the food product by immersing the food, or by spraying or mixing, and although the percentage of the additive is not of great importance, it is generally selected from a range of 0 to about 20 parts by weight per 100 parts by weight of the composition of the present invention.

The food product is any one or more of a fruit, a vegetable, a dried or cut product made from fruits or vegetables, fruit juice, vegetable juice, a mixed juice, or a chip, a noodle, a processed stock farm product, a processed fish product, a dairy product, a fermented dairy product, a bean product, a grain product, a fermented product, a baked product, a condiment, a processed meat product, an acidic beverage, a licorice, and a herb.

Also, the present invention provides a health functional food that contains a shikonin compound effective for preventing and improving diabetes and bromatologically acceptable food additives.

The shikonin compound includes isobutyryl shikonin, β,β-dimethylacryl shikonin, isovaleryl shikonin, or α-methyl-n-butyryl shikonin.

The composition including the compound of the present invention can be used in a variety of ways in pharmaceuticals, foods, and drinks, etc., that are effective in preventing and improving diabetes. Foods to which the compound of the present invention can be added include, for example, various food products, beverages, gums, teas, vitamin complexes, health care foods, etc., and can be used in the form of a powder, grains, tablets, capsules, or beverages.

The compound of the present invention is itself virtually free of toxicity and side affects, and thus can be used safely even when applied for long periods of time for prevention purposes.

The compound of the present invention can be added to a food or drink product for the purposes of preventing diabetes. Here, the amount of the extract in the food or drink product may generally range from 0.01 to 15 weight % of the total weight of the food for a health food composition, and from 0.02 to 10 g, preferably 0.3 to 1 g, per 100 ml for a health drink composition.

Besides including the above compound as an active ingredient in the percentage indicated above, the health drink composition does not include any particular limitations on the liquid component and can include additional ingredients, such as various flavorings or natural carbohydrates, etc., as is common in typical beverages. Examples of natural carbohydrates include common sugars, including monosaccharides, such as glucose, fructose, etc., disaccharides, such as maltose, sucrose, etc., and polysaccharides, such as dextrin, cyclodextrin, etc., as well as sugar alcohols, such as xylitol, sorbitol, erythritol, etc. Besides the above, other flavorings can advantageously be used, including natural flavorings (thaumatin, stevia extracts—such as rebaudioside A, glycyrrhizin, etc.) and synthetic flavorings (saccharin, aspartame, etc.) The content of the natural carbohydrates is generally about 1 to 20 g, preferably about 5 to 12 g, per 100 ml of the composition of the present invention.

In addition to the above, the composition of the present invention can include various nutrients, vitamins, minerals (electrolyte), flavoring agents such as synthetic flavoring agents and natural flavoring agents, coloring agents and improving agents (cheese, chocolate, etc.), pectic acid and salts thereof, alginic acid and salts thereof, organic acids, protective colloidal thickening agents, pH controlling agents, stabilizing agents, preservatives, glycerin, alcohol, carbonizing agents as used in carbonated beverages, etc. Moreover, the compositions of the present invention can include fruit, as used in preparing natural fruit juices and fruit juice beverages and vegetable beverages. These components can be used independently or in combination. Although the percentage of the additive is not of great importance, it is generally selected from a range of 0 to about 20 parts by weight per 100 parts by weight of the composition of the present invention.

It will be apparent to those of ordinary skill in the art that changes can be made to the materials and methods without departing from the spirit and scope of the present invention.

As described above, the shikonin compounds of the present invention exhibit antidiabetic effects by blocking the K_{ATP} ion channels (ATP-sensitive potassium ion channels) of beta (β) cells in the pancreas and increasing the concentration of calcium ions to promote the secretion of insulin, and thus can be used as medicinal pharmaceuticals, food additives, or health functional foods for preventing and treating diabetes mellitus.

### BRIEF DESCRIPTION OF THE DRAWINGS

FIG. 1 illustrates a method of extracting and fractionating Lithospermum erythrorhizon;

FIG. 2 illustrates HPLC results for the Lithospermum erythrorhizon extract;

FIG. 3 illustrates the effects of the Lithospermum erythrorhizon extract on K_{ATP} ion channels for various concentrations of the Lithospermum erythrorhizon extract;

FIG. 4 compares the effects on K_{ATP} ion channels of the Lithospermum erythrorhizon extract with those of GBC, which is a K_{ATP} ion channel inhibitor;

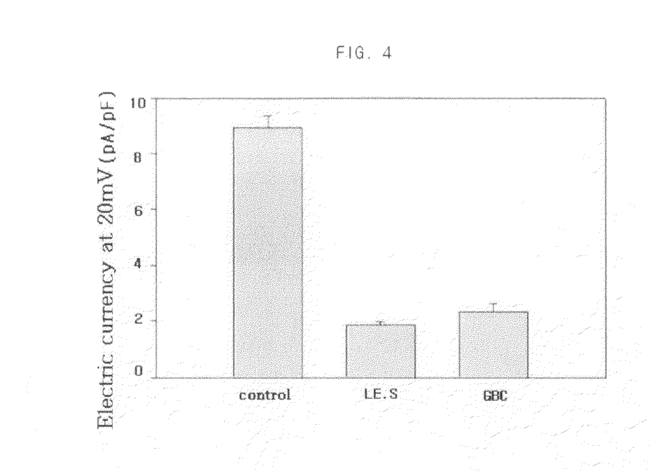

FIG. 5 illustrates the effect of the Lithospermum erythrorhizon extract and the shikonin compounds on K_{ATP} ion channels;

FIG. 6 illustrates the efficacy of the Lithospermum erythrorhizon extract in increasing mRNA expression in proinsulin;

FIG. 7 illustrates the inhibitory effect of the Lithospermum erythrorhizon extract on glucose absorption; and

FIG. 8 illustrates the effect of the Lithospermum erythrorhizon extract on insulin-resistant type II diabetes.

### DESCRIPTION OF THE PREFERRED EMBODIMENTS

The present invention will be described in more detail with reference to the examples below, which illustrate the preparation and utility of the extract and compounds of the present invention.

It will be apparent to those skilled in the art that various modifications and variations can be made to certain materials and methods without departing from the spirit or scope of the present invention.

A detailed description of the present invention is provided below with reference to particular examples and experiments.

However, it should be understood that the following examples and experiments are provided only for illustrative purposes and that the present invention is not to be limited to these examples and experiments in any manner.

### Example 1

### Preparation of the Lithospermum Erythrorhizon Extract

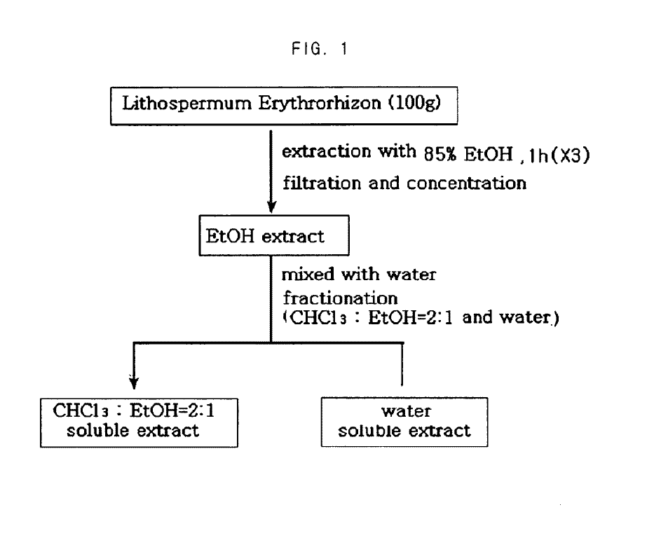

1 kg of Lithospermum erythrorhizon purchased from Kyung-dong market located in Seoul were washed, ground into sizes of 1 cm, dried in a freeze dryer, and kept at −20° C. in a refrigerator. 100 g of the dried sample was subjected to ultrasonic extraction using 1,000 g of 85% ethanol for 1 hour, to obtain 350 g of a Lithospermum erythrorhizon extract (FIG. 1).

### Example 2

### Purification and Isolation of Shikonin Compounds

For the 350 g of Lithospermum erythrorhizon extract obtained in Example 1, the organic solvent was dried under reduced pressure, and the residue was suspended in water and, using a chloroform-ethanol (2:1) solution and purified water, was fractionated into a water layer and a chloroform-ethanol fraction, where HPLC was performed for the chloroform-ethanol (2:1) fraction. The analysis conditions for the HPLC include flowing 60% acetonitrile and 40% distilled water through the column during the 0 to 15 minute period, and then gradually increasing the amount of acetonitrile to 100% during the 15 to 30 minute period. The percentage of acetonitrile was maintained at 100% during the 30 to 40 minute period but was lowered again to 60% after the 40 minute mark.

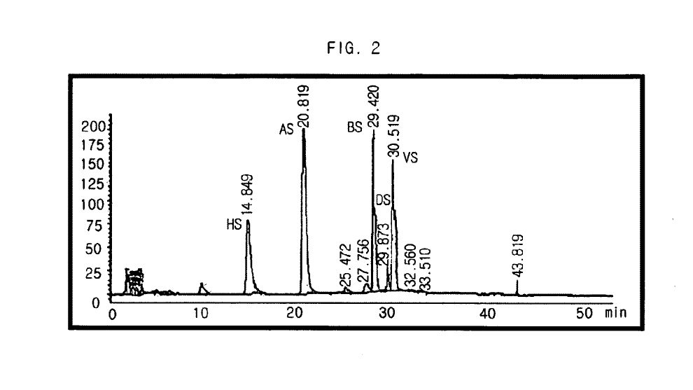

Using semi-preparative HPLC under the analysis conditions described above, the components were isolated to obtain compounds represented by the following chemical formulas 1 to 4 (FIG. 2).

2-1. Isobutyryl Shikonin (Nacalai Tesque INC., Kyoto, Japan) (1)
Molecular Weight: 358.39
Molecular Formula: C₂₀H₂₂O₆
Purity: ≧98% (HPLC)

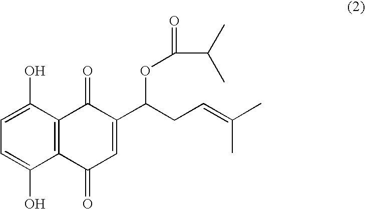

2-2. β. β-dimethylacryl Shikonin (Nacalai Tesque INC., Kyoto, Japan) (2)
Molecular Weight: 370.40
Molecular Formula: C₂₁H₂₂O₆
Purity: ≧98% (HPLC)

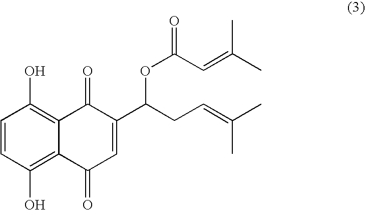

2-3. Isovaleryl Shikonin (Nacalai Tesque INC., Kyoto, Japan) (3)
Molecular Weight: 372.41
Molecular Formula: C₂₁H₂₄O₆
Purity: ≧98% (HPLC)

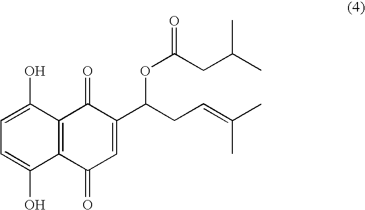

2-4. α-methyl-n-butyryl Shikonin (Nacalai Tesque INC., Kyoto, Japan) (4)
Molecular Weight: 372.41
Molecular Formula: C₂₁H₂₄O₆
Purity: ≧98% (HPLC)

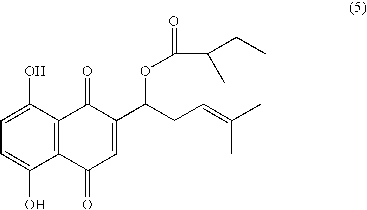

### Reference Example 1

### Cultivation of HIT-T15 Cells

HIT-T15 cells used for the experimental examples below were cultivated in an RPMI 1640 culture medium containing horse serum (10%, v/v), fetal bovine serum (2.5% v/v), and 1% penicillin-streptomycin, in a 5% CO₂ incubator at 37° C.

### Experimental Example 1

### Electro-Physiological Experiment

Since type II diabetes is directly related to the K_{ATP} ion channels of beta cells in the pancreas (Tarasov, A. et al., Diabetes., 53(3), pp. S113-S122, 2004), it was tested whether or not the Lithospermum erythrorhizon extract obtained in the above example or the shikonin compounds isolated therefrom blocks the K_{ATP} ion channels of beta cells in the pancreas.

1-1. Experiment Method

The ion current was recorded by a conventional whole cell patch clamp method, using a patch clamp amplifier (EPC-9, Heka Elektronik, Lambrecht, Germany). The measurement electrode was pulled from a borosilicate glass capillary using a puller (DMZ-Universal Puller; Dagan Co., USA). The electrode was selected to have a resistance of 6 to 9 MΩ when a solution was filled in the electrode. A cover glass, to which the cells were adhered, was placed on a microscope, and the extra-cellular fluid was allowed to flow, due to gravity, at a speed of up to 0.5 ml/min. When measuring the K_{ATP} electric current, the solution inside the electrode included (in mM): 10 NaCl, 102 KCl, 1 CaCl₂, 1 MgCl₂, 10 HEPES, 0.1 Na₂-ATP, 1 Na₂-GTP, and 10 EGTA (pH 7.2). To utilize the voltage-clamp method in taking records, the capacitance and series resistance of the cell membranes were compensated by 80% or higher, and the low-pass filter was set to 1 kHZ during the experiments. To measure the K_{ATP} electric current, the cell solution was substituted with 110 mM of a barium solution (mM: 110 BaCl₂, 10 HEPES, 10 glucose), after which the membrane potential was fixed at −60 mV, and the resulting introvert current was recorded. The experiment results were stored in a Pentium level IBM computer and analyzed using Pulse/Pulsefit (v8.65) software (Heka Elektronik, Lambrecht, Germany), and all of the experiments were performed at room temperature.

1-2. Effect of the Lithospermum Erythrorhizon (LE.S) Extract on K_{ATP} Ion Channels for Various Concentrations

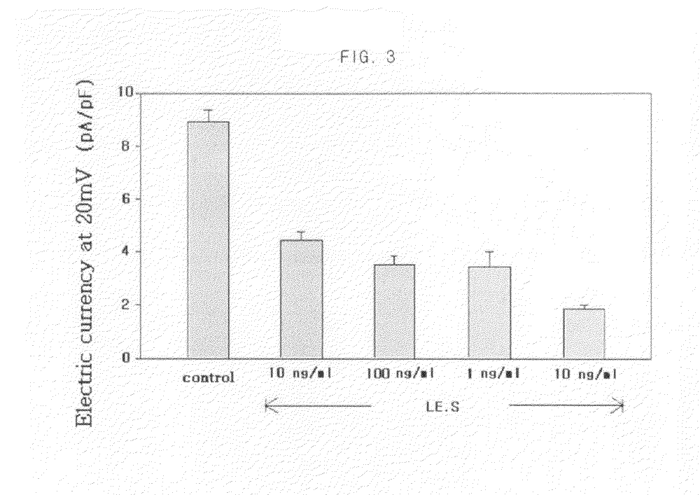

For various concentrations of the Lithospermum erythrorhizon sieb (LE.S) extract, it was observed that the electric current of the K_{ATP} ion channels was reduced from 20 mV according to the concentration. When the LE.S was applied in 10 ng/ml, the current of the ion channels was reduced by about 50%, while 100 ng/ml yielded a reduction of about 66%, and 10 μg/ml yielded a reduction of about 75% (FIG. 3).

1-3. Comparison Between the Effects of the Lithospermum Erythrorhizon (LE.S) Extract and GBC a K_{ATP} Ion Channel Inhibitor, on K_{ATP} Ion Channels

To evaluate how effective the Lithospermum erythrorhizon (LE.S) extract is in inhibiting K_{ATP} ion channels, the effects were compared with those of GBC (glibenclamide), a well-known K_{ATP} ion channel inhibitor, and the results showed that LE.S provide reductions at a level comparable to GBC. It was observed that LE.S provides an efficacy in inhibiting K_{ATP} ion channels more effectively than GBC, which is a K_{ATP} ion channel inhibitor.

1-4. Comparison Between the Effects of the Lithospermum Erythrorhizon Extract and Shikonin Single Compounds on K_{ATP} Ion Channels

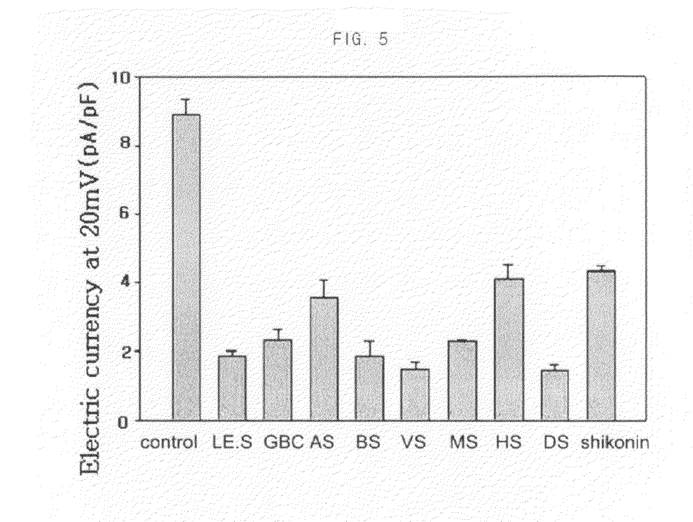

Comparing the Lithospermum erythrorhizon extract and the shikonin single compounds with the K_{ATP} ion channel inhibitor GBC, it was observed that the shikonin single compounds, iso-butyryl shikonin (BS), isovaleryl shikonin (VS), α-methyl-n-butyryl shikonin (MS), and β,β-dimethylacryl shikonin (DS), effectively inhibit K_{ATP} ion channels. It was observed that VS and DS in particular are the best in providing the inhibitory effects. However, acetyl shikonin (AS) showed inhibiting levels higher than those of GBC (FIG. 5).

### Experimental Example 2

### Efficacy in Increasing Proinsulin mRNA Expression

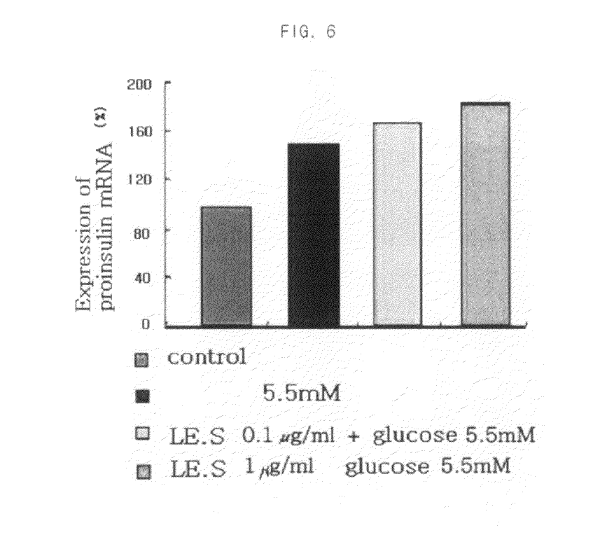

The present experiment was performed to verify that the Lithospermum erythrorhizon extract increases insulin secretion when sugar levels are increased in pancreatic cells. It was observed that, when glucose is applied to HIT-T15 cells, the test groups to which 0.1 μg/ml and 1 μg/ml of the Lithospermum erythrorhizon extract were administered, respectively, showed an increase in proinsulin mRNA expression by 13% and 20%, respectively, compared to the control group (FIG. 5). The pancreas is the main organ for secreting insulin, and an increase in the mRNA of insulin precursors increases insulin secretion. Therefore, when there is an increase in glucose, the Lithospermum erythrorhizon extract causes an increase in insulin secretion, to lower the glucose levels (FIG. 6).

### Experimental Example 3

### Sugar Loading Antidiabetic Experiment

Seven-week old male ICR mice were purchased from Chung-ang Experiments Co. Ltd, for use in the experiments. Sucrose was orally administered in an amount of 2.0 g/kg to the mice, which had been starved for 18 hours, where a physiological saline solution was orally administered simultaneously with the sucrose to a control group, while a Lithospermum erythrorhizon active fraction sample was orally administered simultaneously with the sucrose. After a designated period of time (0, 15, 30, 60, 120 minutes) blood samples were collected from the tail veins, and the blood sugar levels were measured using a Onetouch Ultra Glucose Test Kit (LifeScan, Inc. U.S.A.). The results of the experiments were represented in the form of mean±standard deviation, and the significance of the results was verified by a student t-test (**p<0.01, n=5).

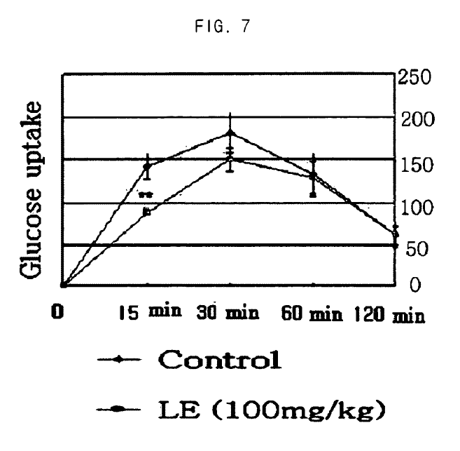

The experiment results show a normal glucose kinetic profile, as the maximum blood sugar level was reached at 30 minutes after the sucrose loading. The blood sugar levels of the mice to which the Lithospermum erythrorhizon extract was administered at 15, 30, 60, and 120 minutes after the sucrose loading, were lower by 39%, 18%, 4%, and 1%, respectively, compared to the blood sugar levels of normal mice, with the inhibitory effects against sugar absorption especially high for the cases of 15 minutes and 30 minutes. As such, the effects of inhibiting initial sugar absorption during food intake confirm their usage as dietary or antidiabetic remedies for diabetes patients (FIG. 7).

### Experimental Example 4

### Effect of the Lithospermum Erythrorhizon Extract (LE) on Type II Diabetes

After intraperitoneally administering the Lithospermum erythrorhizon extract (LE) once daily to the db/db mice, blood samples were collected at designated days (0, 1, 2, 3, and 4 days) from the tail veins and measured using a Onetouch Ultra Glucose Test Kit (LifeScan, Inc. U.S.A.).

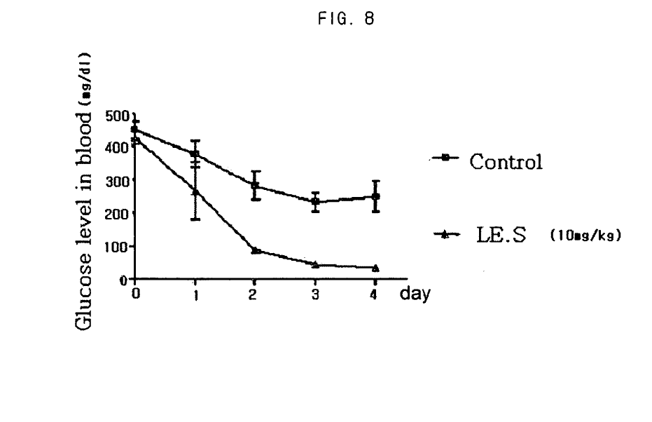

It was observed that when the Lithospermum erythrorhizon extract fraction (LE) was intraperitoneally administered to the db/db mice once every day, the levels decreased with the passage of time more than the control group. In particular, it was observed that the glucose levels in the serum were effectively lowered at 3 days and 4 days after administering the Lithospermum erythrorhizon extract fractions (FIG. 8).

### Pharmaceutical Example 1

### Preparation of a Powder

α-methyl-butyryl shikonin 20 mg

Lactose 100 mg

Talc 10 mg

The above components were mixed and filled in a sealed package to prepare a powder.

### Pharmaceutical Example 2

### Preparation of a Tablet

Isobutyryl shikonin 10 mg

Corn Starch 100 mg

Lactose 100 mg

Magnesium stearate 2 mg

The above components were mixed and cast to prepare a tablet.

### Pharmaceutical Example 3

### Preparation of a Capsule

β,β-dimethylacryl shikonin 10 mg

Crystalline cellulose 3 mg

Lactose 14.8 mg

Magnesium stearate 0.2 mg

The above components were mixed and filled in a gelatin capsule according to a typical method for preparing capsules.

### Pharmaceutical Example 4

### Preparation of an Injection

Isovaleryl shikonin 10 mg

Mannitol 180 mg

Sterilized distilled water 2974 mg

Na₂HPO₄12H₂O 26 mg

The above components were included in the above amounts for 1 ampoule (2 ml) according to a typical method for preparing injections.

### Pharmaceutical Example 5

### Preparation of a Liquid

α-methyl-n-butyryl shikonin 20 mg

Isomerized sugar 10 g

Mannitol 5 g

Purified water suitable amount

Each component was added to and dissolved in purified water, a suitable amount of a lemon flavoring was added, and the above components were mixed, after which purified water was added to adjust the overall volume to 100 ml, and the mixture was filled in a brown bottle and sterilized to prepare a liquid.

### Pharmaceutical Example 6

### Preparation of a Health Food Product

β,β-dimethylacryl shikonin 1,000 mg

Vitamin mixture suitable amount

Vitamin A acetate 70 μg

Vitamin E 1.0 mg

Vitamin B₁ 0.13 mg

Vitamin B₂ 0.15 mg

Vitamin B₆ 0.5 mg

Vitamin B₁₂ 0.2 mg

Vitamin C 10 mg

Biotin 10 μg

Nicotinic acid amide 1.7 mg

Folic acid 50 μg

Calcium pantothenate 0.5 mg

Mineral mixture suitable amount

Ferrous sulfate 1.75 mg

Zinc oxide 0.82 mg

Magnesium carbonate 25.3 mg

Monocalcium phosphate 15 mg

Calcium hydrogen phosphate 55 mg

Potassium citrate 90 mg

Calcium carbonate 100 mg

Magnesium chloride 24.8 mg

While the content ratio of the above vitamin and mineral mixture is for a preferred example relatively suitable for a health food product, it is possible to change the ratio. According to a typical method for preparing health food products, the above components can be mixed and prepared as grains, which can be used to prepare a health food composition according to typical methods.

### Pharmaceutical Example 7

### Preparation of a Health Drink Product

Isobutyryl shikonin 100 mg

Vitamin C 15 g

Vitamin E (powder) 100 g

Ferrous lactate 19.75 g

Zinc oxide 3.5 g

Nicotinic acid amide 3.5 g

Vitamin A 0.2 g

Vitamin B₁ 0.25 g

Vitamin B₂ 0.3 g

Water suitable amount

According to a typical method for preparing health drink products, the above components can be mixed and then stirred and heated for about 1 hour at 85° C., after which the solution was filtered and collected in a sterilized 2 l container, sealed and sterilized, and kept refrigerated, to be used in preparing a health drink composition.

While the content ratio is for a preferred example relatively suitable for a drink product, it is possible to change the ratio according to geographic and ethnic preferences, such as consumer class, consumer nation, usage, etc.

As described above, the composition of the present invention showed potent antidiabetic activity and safety therefore it can be used as the therapeutics, health functional food or food additive for treating and preventing diabetes mellitus.

1. A pharmaceutical composition for the prevention and treatment of diabetes mellitus, the pharmaceutical composition comprising a shikonin compound represented by general chemical formula (1) below as an active ingredient, and a pharmaceutically acceptable carrier, diluent, or excipient: 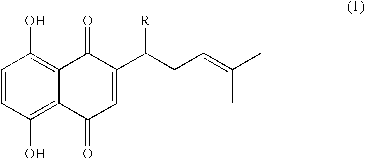
wherein,
R is a member of the group consisting of OCOCH(CH₃)₂, OCOCH═C(CH₃)₂, OCOCH₂CH(CH₃)₂, and OCOCH(CH₃)CH₂CH₃.

2. The pharmaceutical composition according to claim 1 wherein the shikonin compound is selected from isobutyryl shikonin, β,β-dimethylacryl shikonin, isovaleryl shikonin or α-methyl-n-butyryl shikonin.

3. A use of a shikonin compound represented by the general chemical formula (1) as set forth in claim 1, for the preparation of a therapeutic agent for the treatment and prevention of diabetes mellitus.

4. A method of treating or preventing diabetes mellitus by administering an effective amount of the shikonin compound represented by the general chemical formula (1) as set forth in claim 1 and a pharmaceutically acceptable carrier or excipient to a human or a mammal.

5. A food additive comprising a shikonin compound represented by the general chemical formula (1) as set forth in claim 1, for the prevention and improvement of diabetes mellitus.

6. The food additive according to claim 5, wherein the health food is provided in a powder, granular, tablet, capsule or liquid form.

7. The food additive according to claim 5, wherein the shikonin compound is selected from isobutyryl shikonin, β,β-dimethylacryl shikonin, isovaleryl shikonin or α-methyl-n-butyryl shikonin.

8. A health functional food comprising a shikonin compound represented by the general chemical formula (1) as set forth in claim 1 as an active ingredient for the prevention and improvement of diabetes mellitus.

9. The health functional food according to claim 8, wherein the shikonin compound is selected from isobutyryl shikonin, β,β-dimethylacryl shikonin, isovaleryl shikonin or α-methyl-n-butyryl shikonin.
